(19) United States
(12) Patent Application Publication
(10) Pub. No.: US 2012/0016337 A1
(43) Pub. Date: Jan. 19, 2012

(54) Collection canister apparatus for closed loop fat harvesting system and method of use thereof

(76) Inventor: Ben M. Khalaj, (US)

(21) Appl. No.: 13/135,746

(22) Filed: Jul. 14, 2011

Related U.S. Application Data
(60) Provisional application No. 61/399,664, filed on Jul. 15, 2010.

(51) Int. Cl. A61M 5/31; A61M 1/00

(52) U.S. Cl. 604/506; 604/542

(57) ABSTRACT

An improved collection canister and method of its use for a closed loop fat harvesting system is disclosed. The collection canister comprising of a main body with inner cavity, handles on each sides, a detachable lid that includes at least two liposuction lure ports, a vacuum port to provide suction within the main body and an additional port for adding additive via a syringe as needed to treat the fat cells such as growth factors. Furthermore, the collection canister has an open/close valve for draining and withdrawing fat cells into syringes.

Applicant claim priority-filling date for this application. Provisional application filed on Jul. 15, 2010, and the application No. 61/399,664.

### FIELD OF THE INVENTION

The present invention relates to a canister that collects fat cells through liposuction in a closed loop fat harvesting system and transplantation thereof. More particularly the canister can accommodate large volume of fat cells and provides multiple withdrawal for different sizes of syringes.

### BACKGROUND OF THE INVENTION

Fat transfer is a medical procedure that uses the patient's own fat tissue to increase the volume of fat in the subcutaneous area of the body. In recent years, the process of harvesting fat through liposuction and transferring it to different area of body's part such as breast augmentation is a common procedure in plastic surgery. In such medical procedure, transferring of the fat cells from starting point of extracting the fat and collecting it and then injecting it into patient's body is curial operation with respect to care of handling the fat cells. Such handling is considered in closed loop fat transfer system. In this system, surgeon find a donor area of a patient's body like lower stomach or inner thighs and using liposuction technique to extract the fat cells. The extracted fat cells directed to collection container such as a canister or a large medical syringe. Surgeon uses smaller size syringes to withdraw fat cells from the large syringe to be applied to applicable area of patient's body.

Although, the current method of collecting fat cells can work with respect to closed loop fat transfer, but there are some disadvantages and limitations due to collecting output, treating the fat cells and disseminating the fat cells for injection proposes.

However, this invention overcomes the shortcomings and solving the problems associated with prior art. This invention has improved the collection canister apparatus for fat transfer and method of handling the fat cells thereof.

### SUMMARY OF THE INVENTION

It is an object of the present invention to provide a canister that collects fat cells through liposuction via a closed loop fat harvesting system. The collection canister comprising of a main body with inner cavity, a pair of handles on each sides, a detachable lid that includes at least two liposuction lure ports, a vacuum port to provide suction within the main body and an additional port for adding additive via a syringe as needed to treat the fat cells such as growth factors known as PRP (Platelet Rich Plasma). Furthermore, the collection canister has an open/close valve for draining and withdrawing fat cells into syringes.

It is also an object of this invention to mount the collection canister via its handles to a vertical post bracket. The canister position can be adjusted with respect to vertical post.

It is yet another object of this invention to mount the collection canister via its base to a table mount bracket. The canister position is fixed with respect to the table.

It is further object of this invention to provide a method of using the canister by importing the fat cells into cavity of main body of the canister, draining the unwanted fluid, treating the fat cell with additive such as growth factors, mixing the fat cells with additive and withdrawing the fat cells into multiple different sizes of syringes for propose of injecting into patient's body.

Further objects and advantages of this invention will become apparent from consideration of the drawings and descriptions that follow.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a top perspective view of a closed loop fat harvesting system showing the present invention in conjunction with the collection canister.

FIG. 2 is a top perspective view of the collection canister.

FIG. 3 is an exploded view of collection canister.

FIG. 4 is a top view of the collection canister lid.

FIG. 5 is a bottom perspective view of the collection canister lid.

FIG. 6 is a perspective view of the importing fat cells into the canister main body.

FIG. 7 is similar view like FIG. 6, but the drain valve is open position to drain unwanted fluid from liposuction extraction.

FIG. 8 is similar view like FIG. 7, but the valve is in closed position and optional additive is injecting onto the fat cells in main body.

FIG. 9 is similar view like FIG. 8, but the valve is in open position and syringe connected to the sterile tube to provide air to mix the fat cells with injected additive.

FIG. 10 is similar view like FIG. 9, but the syringe is withdrawing treated fat cells for propose of injecting into patient body.

### DETAILED DESCRIPTION OF THE INVENTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

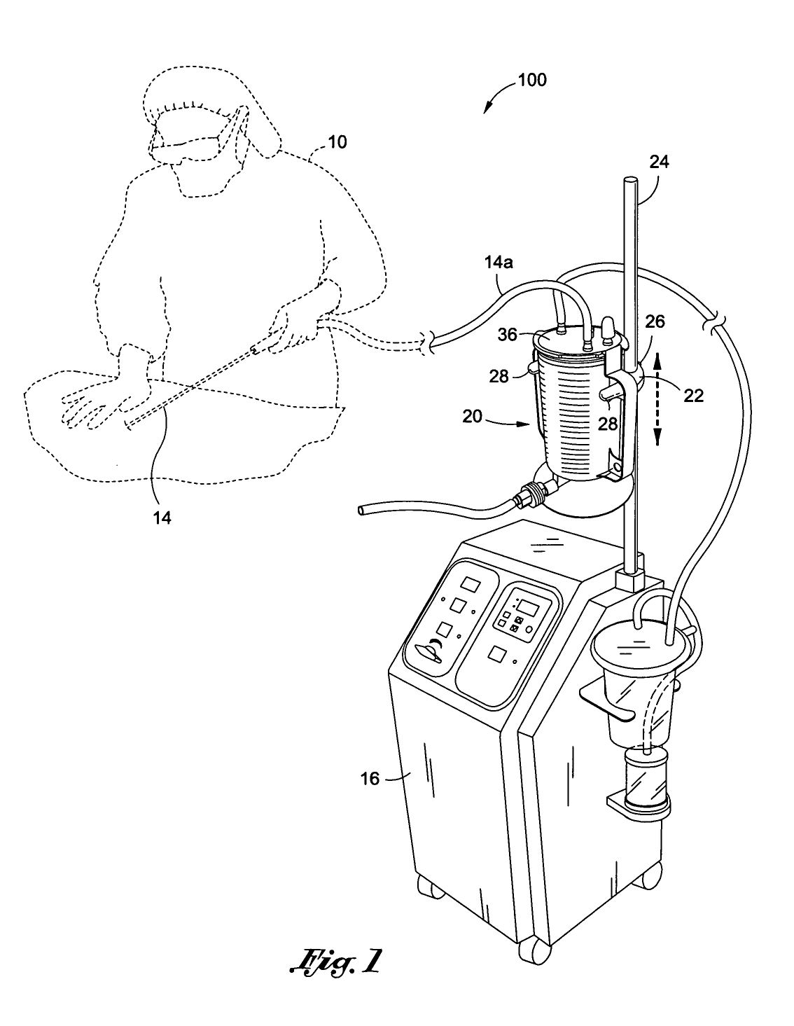

FIG. 1 is illustrating a preferred embodiment of a closed loop fat harvesting system 100 showing the present invention in conjunction with the collection canister 20. In this system, a surgeon 10 extract fat cells 12 from a patient through liposuction cannula 14 via a vacuum source from aspirator console 16. The fat cells directed toward the collection canister 20 via an sterile tubing 14a.

The collection canister 20 detachably is mounted on a vertical post 24 by intermediate mounting bracket 22 and fastener nuts 28 and can be adjusted in vertical positioning using a set screw 26 as shown in FIG. 1. Furthermore, the canister 20 can also be mounted by its base 20c to a table mounted fixture.

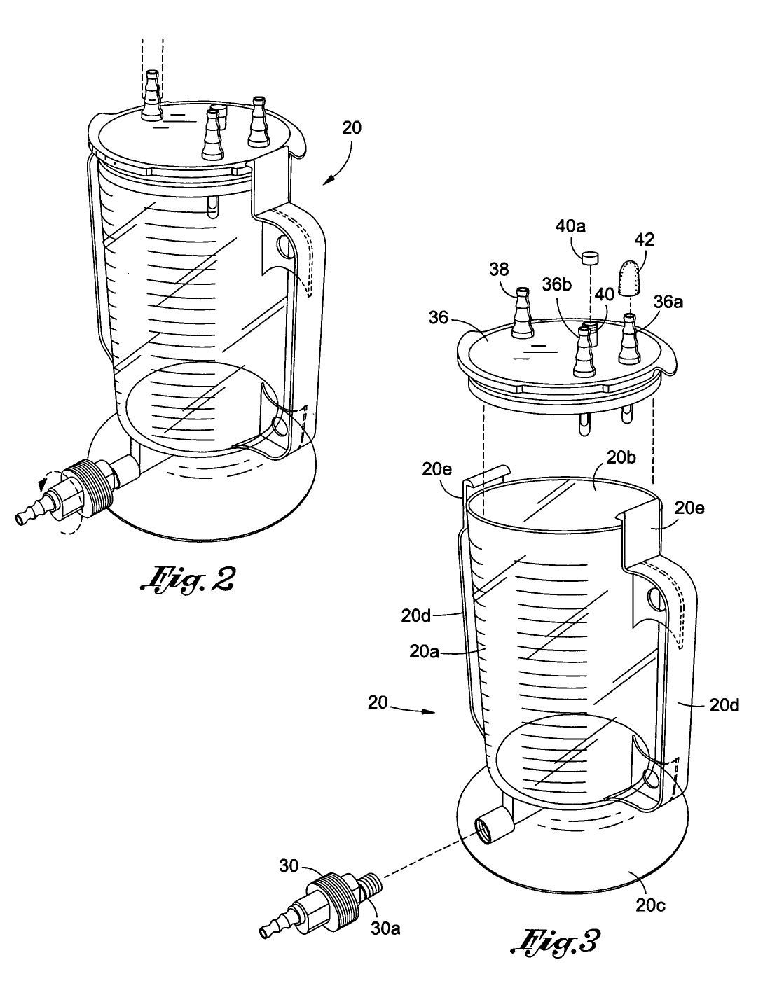

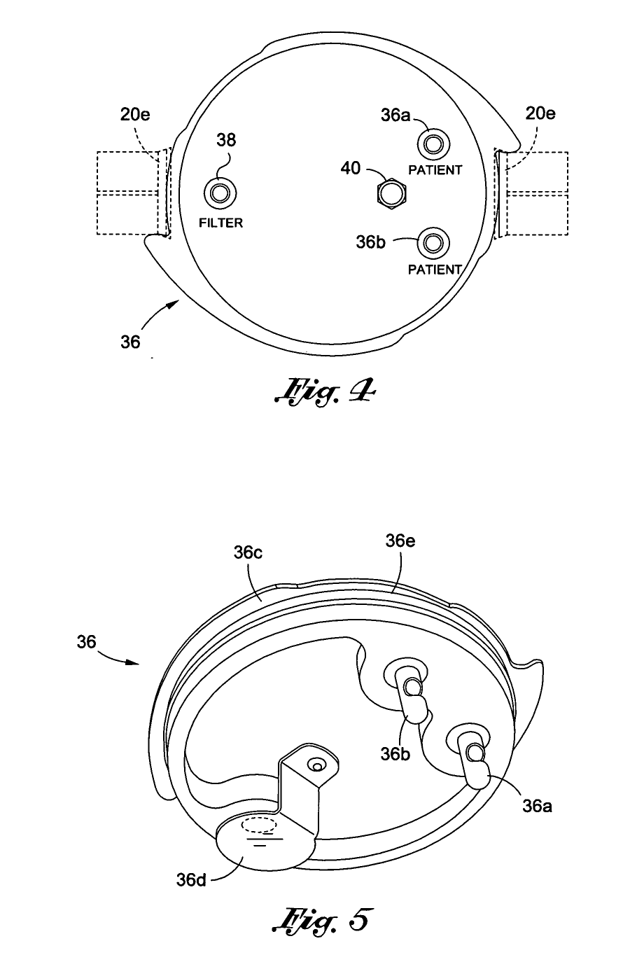

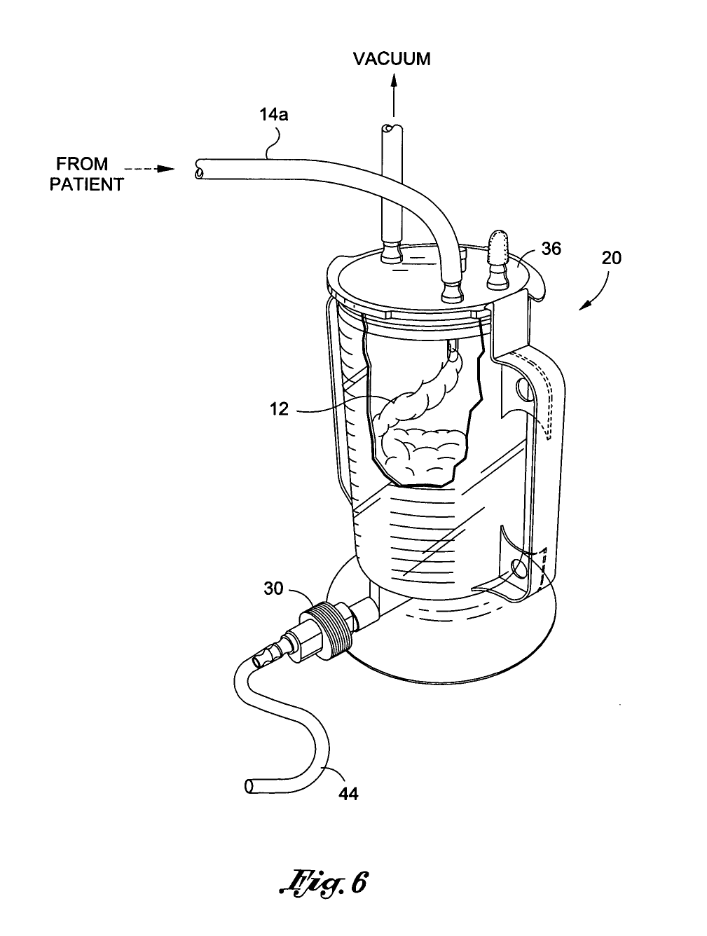

The collection canister 20 comprising a main body 20a with inner cavity 20b, a pair of handles 20d, a detachable lid 36, and a base 20c as shown in FIG. 2-5. The lid 36 includes two liposuction ports 36a, 36b, a suction port 38 and a port 40 for adding additives. This port 40 has a threaded cap 40a to block the port when it is not being used. Furthermore, it has an o-ring 36e to prevent any leakage within the inner cavity 20b of main body 20a. The lid suction port 38 also has a shield underside and placed within a distance of the suction opening to prevent any chunk of fat enters the suction parts and its tubing as seen in FIG. 5.

In order to place the lid 36 into the inner cavity 20b of main body 20a, the locking tab 36c of the lid tucks under the main body hook 20e and by twisting the lid 36 assembly clockwise, the lid attachably is being secured to main body 20a.

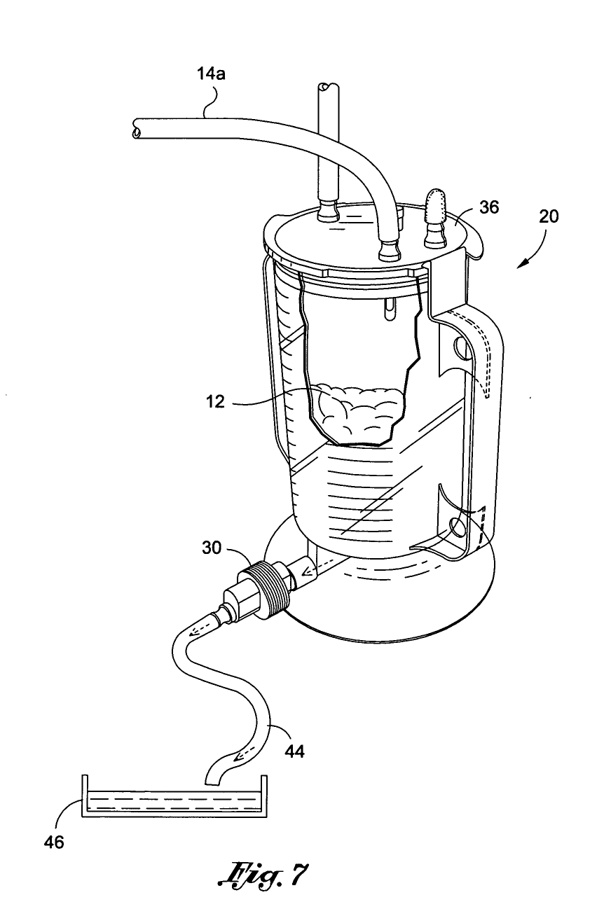

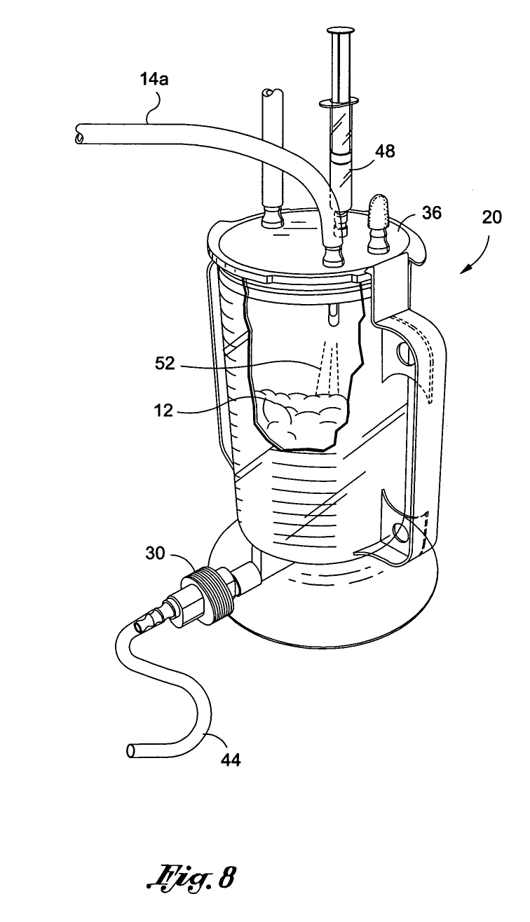

Now refereeing to FIG. 6-10 are showing the preferred method of use for the collection canister 20. Once the desired fat cells volume enters into inner cavity 20b of the collection canister 20, the suction source will be turned off and allowing the fat to separate fat cells from fluid. The next process is to drain the fluid as shown in FIG. 7. By opening the drain valve 30, the fluid within the inner cavity 20b flows down with gravitation force into the sterile silicon tube 44 and collected it into basin 46.

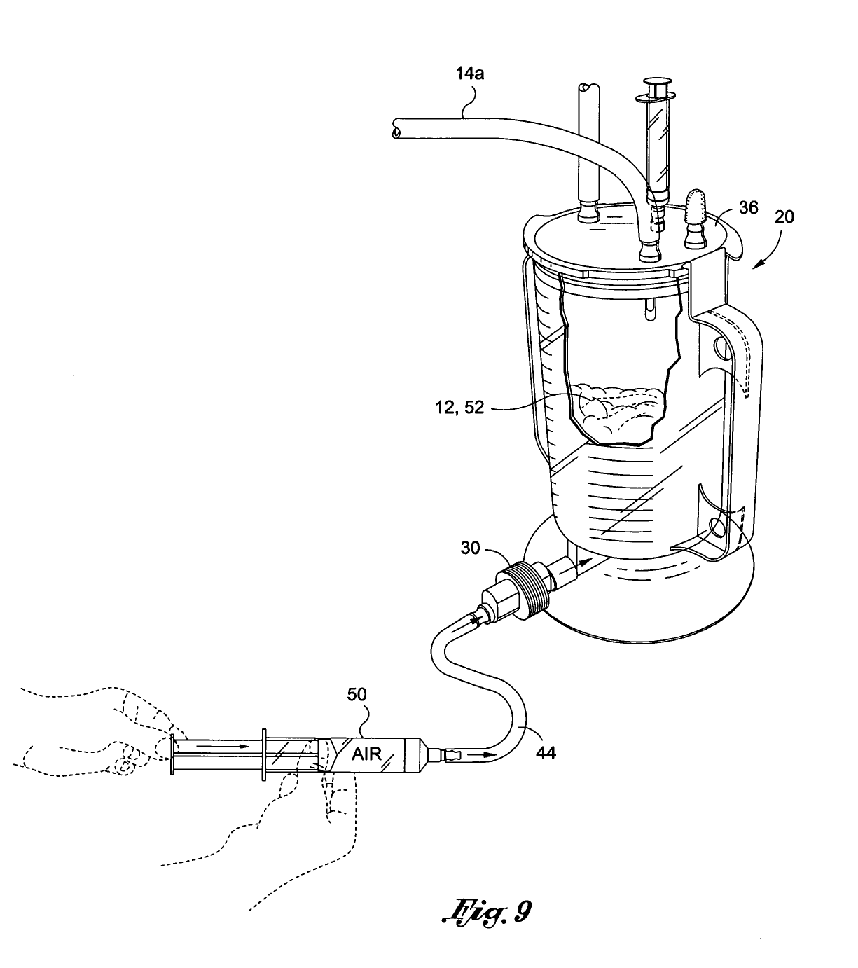

The next process is to add additives for the fat cells such as cells growth factors known as PRP (Platelet Rich Plasma) 52 injected onto the fat cells 12 via a syringe 48 using the port 40, as shown in FIG. 8. Plastic surgeon use the growth factor along with the extracted fat cells to bring stability to injected area of patient body. The dose needed for this process is determined by operating surgeon with respect to amount of fat the collected in inner cavity.

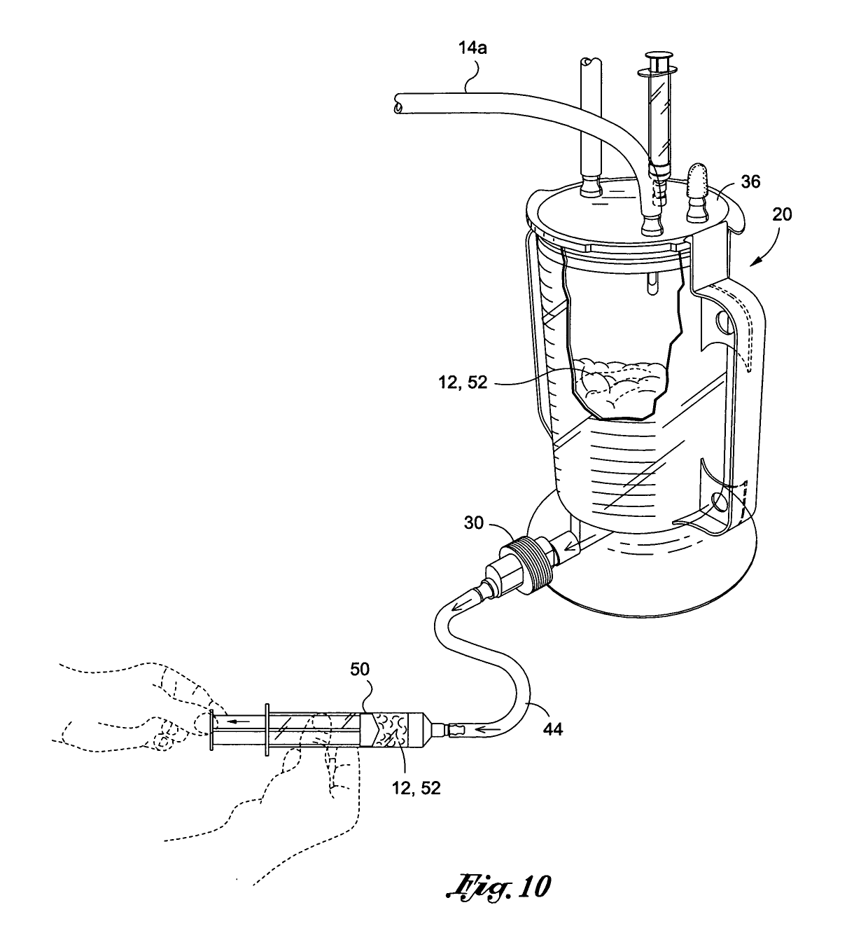

Following step in process is to mix the fat cells with the added additives. In order to do this, surgeon connect Tommy syringe 50 full of air to the tubing 44 and open the valve 30 and inject air into the inner cavity 20b to blend the fat cells with additives. This process may be repeated to get a satisfactory result of the mixture as shown in FIG. 9.

Once the mixture is ready, the Tommy syringe 50, withdraw the fat cells from the collection canister 20 via the sterile silicon tubing 44 as illustrated in FIG. 10. Some different sizes of syringes can be use in this configuration using an adopted female lure that connected to end of silicon tubing 44.

While this invention is susceptible of embodiments in many different forms, this specification and the accompanying drawings disclose only some specific forms as examples of the invention. The invention is not intended to be limited to the embodiments so described; however, the scope of the invention is pointed out in the appended claims

I Claim:

1. A collection canister in conjunction with a closed loop fat harvesting system comprising:
a main body, said main body has inner cavity for collection of fat cells, said main body has a pair of handles on each sides of said main body, said main body also has a base and a drain hole next to said main body base,
a detachable lid, said lid fits tightly on the top of said main body, said lids includes at least two liposuction lure ports, a vacuum port to provide suction within the said main body and a port for adding additive to treat the fat cells, and;
an open/close valve, said valve connects to said main body drain hole, said valve is for draining fluid and withdrawing fat cells into syringes.

2. A collection canister according to claim 1, wherein said a pair of handles, said handles includes mounting holes.

3. A collection canister according to claim 2, wherein said mounting holes, said mounting holes connects to adjustable bracket on a vertical post.

4. A collection canister according to claim 1, wherein said a base, said base can be mounted to a table.

5. A collection canister according to claim 1, wherein said a additive, said additive is cell growth factors known as PRP.

6. A method of use for collection canister in conjunction with a closed loop fat harvesting system comprising:
importing the fat cells into cavity of main body of the canister via the liposuction lure ports,
draining the unwanted fluid,
treating the fat cell with additive,
mixing the fat cells with said additive, and;
withdrawing the fat cells into a syringe for propose of injecting into patient's body.

7. A method of use for collection canister according to claim 6, wherein said a syringe, can be different sizes of syringes.
